(19) United States
(12) Patent Application Publication
(10) Pub. No.: US 2025/0002478 A1
(43) Pub. Date: Jan. 2, 2025

(54) MODIFIED INDOLE COMPOUNDS

(71) Applicant: Psilera Inc., Tampa, FL (US)

(72) Inventors: Jeanine Yacoub, Lakeland, FL (US); Elena Bray, Temple Terrace, FL (US); Jacqueline Salm, Tampa, FL (US); Christopher Witowski, Tampa, FL (US)

(21) Appl. No.: 18/689,983

(22) PCT Filed: Oct. 24, 2022
(86) PCT No.: PCT/US2022/047552

§ 371 Date: Mar. 7, 2024

Related U.S. Application Data
(60) Provisional application No. 63/273,710, filed on Oct. 29, 2021.

(51) Int. Cl. C07D 403/04; A61K 31/4045; A61K 31/4155; A61K 31/4192; C07D 209/16

(52) U.S. Cl. CPC C07D 403/04; A61K 31/4045; A61K 31/4155; A61K 31/4192; C07D 209/16

(57) ABSTRACT

Novel compounds are provided. The compounds are modified at the four position of indole and are structurally related to DMT and DMT derivatives. These compounds may be used in applications such as those in which DMT compounds and compounds structurally related to DMT are used.

### CROSS REFERENCE TO RELATED APPLICATION

This application is a national stage application of PCT/US2022/047552, filed Oct. 24, 2022, which claims the benefit of both the filing date of claims the benefit of the filing date of U.S. provisional patent application Ser. No. 63/273,710, filed Oct. 29, 2021, the entire disclosures of which are incorporated by reference.

### FIELD OF THE INVENTION

The present invention relates to modified indole compounds.

### BACKGROUND OF THE INVENTION

Psychoactive drugs are compounds that affect behavior, mood, thoughts, or perception. These drugs include antipsychotics, anti-anxiety agents, stimulants, reuptake inhibitors, monoamine oxidase inhibitors (MAOI), tricyclic antidepressants, and mood stabilizers. Some of these compounds have historically been used for off label psychoactive activity and are now being investigated for positive clinical efficacy. Many of these compounds are derived from indole.

N,N-dimethyltryptamine (DMT), which is a derivative of indole, and certain other compounds that are structurally related to DMT have recently been reported as being of value when administered to subjects. However, the number of possible modifications of indole compounds is infinite. Thus, there is a need to develop new compounds that are derivatives of indole.

### SUMMARY OF THE INVENTION

The present invention is directed to new indole derived compounds (also referred to as modified indole compounds), methods for making these new compounds, and methods of using these new compounds. Structurally, these compounds differ from DMT compounds and derivatives of DMT compounds by including certain moieties at the indole 4 position.

According to a first embodiment, the present invention provides a compound of formula I:

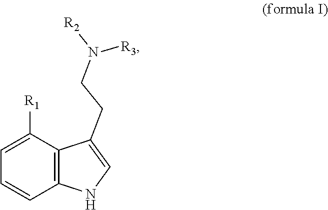

wherein
R₁=COOH, CONH₂, CH₂OH, CH₂OCOCH₃, CH₂OCH₃, CH₂NH₂, CH₂CONH₂, CH₂NHSO₂CH₃, pyrazol, triazol, tetrazol, imidazol, or other nitrogen-containing, 5-membered heterocycles attached at either a carbon or nitrogen atom;
R₂=H or CₙH₂ₙ₊₁, wherein n=1 to 12; and
R₃=H or CₘH₂ₘ₊₁, wherein m=1 to 12.

According to a second embodiment, the present invention provides a formulation or medicament that comprises a compound of formula I and one or more excipients.

According to a third embodiment, the present invention provides use of a compound of formula I, wherein the use is the treatment, management, or prevention of a neurological, mood, or abuse disorder or disease. The disorder may, for example, be depression, central nervous system inflammation, addiction, headache, or dementia, or a disorder of cognition and memory.

According to a fourth embodiment, the present invention provides a method for making a compound of formula I.

### DETAILED DESCRIPTION OF THE INVENTION

Reference will now be made in detail to various embodiments of the present invention. In the following description, numerous specific details are set forth in order to provide a thorough understanding of the present invention. However, unless otherwise indicated or implicit from context, the details are intended to be examples and should not be deemed to limit the scope of the invention in any way. Additionally, features described in connection with the various or specific embodiments are not to be construed as not appropriate for use in connection with other embodiments disclosed herein unless such exclusivity is explicitly stated or implicit from context.

Headers are provided herein for the convenience of the reader and do not limit the scope of any of the embodiments disclosed herein.

### Definitions

Unless otherwise stated or implicit from context the following terms and phrases have the meanings provided below.

The indefinite articles “a” and “an” and the definite article “the” include plural as well as singular referents, unless the context clearly dictates otherwise.

The terms “about” and “approximately” mean an acceptable error for a particular value as determined by one of ordinary skill in the art, which depends in part on how the value is measured or determined. In certain embodiments, the term “about” or approximately” means within 1, 2, 3, or 4 standard deviations. In certain embodiments, the term “about” or “approximately” means within 30%, 20%, 15%, 10%, 9%, 8%, 7%, 6%, 5%, 4%, 3%, 2%, 1%, 0.5%, 0.1%, or 0.05%, of a given value or range.

“Abuse disorder” refers to a disorder or disease that affects a person's brain and behavior and leads to an inability to control the use of a legal or illegal drug or medication. Prescription medicines, non-prescription medicines, and non-approved drugs may all be abused drugs. Drugs and medication that can be abused include but are not limited to substances such as amphetamines, opioids, benzodiazepines, cocaine, barbiturates, alcohol, marijuana, and nicotine.

The terms “composition” and “formulation” as used herein are intended to encompass compositions comprising the specified ingredient(s) (in the specified amounts, if indicated), as well as any product(s) that result, directly or indirectly, from combination of the specified ingredient(s) in any specified amount(s).

“DMT” refers to N,N-dimethyltryptamine, which is a molecule that has the following structure:

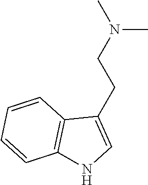

An “excipient” is a substance that may be formulated with an active ingredient of a medication such as a compound of formula I of the present invention. One may include an excipient for any one or more of a number of reasons, including but not limited to long-term stabilization, as bulking agents or to confer a therapeutic enhancement on the active ingredient, such as facilitating absorption, reducing viscosity, or enhancing solubility of an active ingredient. Examples of excipients include but are not limited to: (1) sugar compounds, e.g., lactose, dextrin, glucose, sucrose and sorbitol, and inorganic compounds such as silicates, calcium and magnesium salts, NaCl, and KCl for use as diluents; (2) synthetic polymers, e.g., starches, sugars, sugar alcohols, and cellulose derivatives for use and binders; (3) starch, cellulose derivatives, alginates and crospovidone for use as disintegrants; (4) colloidal anhydrous silicon and other silica compounds for use as glidants; (5) stearic acid and its salts, e.g., magnesium stearate or use as lubricants; (6) sucrose and cellulose acetate phthalate for use as coatings or films; and (7) coloring agents such as synthetic and/or natural dyes.

As used herein “immediate release” is defined as the formulation of an active pharmaceutical ingredient such as a compound of formula I taken orally, nasally or transdermally that results in the rapid absorption of the drug into the blood after administration.

The term indole refers to an aromatic heterocyclic organic compound with a formula of C₈H₇N and a structure of:

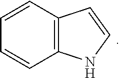

Derivatives of indole have one or more substituents or moieties bound to one or more positions of either ring or each ring. DMT is a derivative of indole.

The terms “manage,” “managing,” and “management” refer to preventing or slowing the progression, spread, or worsening of a disease or disorder, or of one or more symptoms thereof. Often, the beneficial effects that a subject derives from a prophylactic and/or therapeutic agent do not result in a cure of the disease or disorder. In this regard, the term “managing” encompasses treating a subject who had suffered from the particular disease in an attempt to prevent or minimize the recurrence of the disease.

As used herein, “modified release” or “extended release” is defined as a formulation of an active pharmaceutical ingredient such as a compound of formula I taken orally, nasally or transdermally that releases the active pharmaceutical ingredient over several hours or days, to maintain a relatively constant plasma concentration of the drug. These modifications may have a number of objectives, such as maintaining therapeutic activity for an extended time, reducing toxic effects, protecting the active substance against degradation due to low pH, targeting the active substance to a predefined segment of the gastrointestinal tract for local treatment or targeting active substance release at specified time-points.

“Mood disorder” refers to a group of conditions in which a disturbance in a person's mood is the underlying feature. Mood disorders may be groups of mania (elevated mood disorders) or hypomania (depression).

As used herein “oral” relates to a medication in a form for absorption through the oral mucosal, sublingual, buccal, esophageal, gastric, or intestinal membranes. The term “capsule” refers to an oral composition in which the active pharmaceutical ingredient and inactive ingredients are contained as a solid, liquid or semisolid within an outer shell comprised of gelatin, polymerized cellulose, or other suitable material. A capsule is intended to be swallowed wherein the composition will dissolve and release the active pharmaceutical ingredient for systemic absorption through the esophageal, gastric, or intestinal lining.

“Nasal” and “intranasal” refer to being or supplying a medication in a form for absorption through the nasal mucosa. Nasal delivery may be affected through a wide range of forms including but not limited to solutions, gels, suspensions, emulsions, liposomes and microparticles.

“Neurological disorder” refers to diseases of the central and peripheral nervous system e.g., the brain, spinal cord, cranial nerves, peripheral nerves, nerve roots, autonomic nervous system, neuromuscular junction, and muscles. These disorders include epilepsy, Alzheimer's disease and other dementias, cerebrovascular diseases including stroke, migraine, cluster headaches and other headache disorders, multiple sclerosis, Parkinson's disease, neuroinfections, brain tumors, traumatic disorders of the nervous system due to head trauma, and traumatic disorders due to traumatic or terrifying experiences (Posttraumatic Stress Disorder) and neurological disorders as a result of malnutrition and substance abuse. The substance abused may be any number of addictive substances, including but not limited to alcohol and drugs and combinations thereof. Additionally, many bacterial (e.g., Mycobacterial tuberculosis, Neisseria meningitides), viral (e.g., Human Immunodeficiency Virus (HIV), Lyme Disease, Enteroviruses, West Nile Virus, Zika), fungal (e.g., Cryptococcus, Aspergillus), and parasitic (e.g., malaria, Chagas) infections can affect the nervous system and lead to neurological disorders. Neurological symptoms that accompany these disorders may occur because of an infection itself, and/or an immune response.

A “pharmaceutically acceptable salt” is a salt that is of sufficient purity and quality for use in a formulation of a composition or medicament of the present invention. Both human use (clinical and over-the-counter) and veterinary use are included within the scope of the present invention. A formulation of the present invention includes a composition or medicament for either human or veterinary use. Pharmaceutically acceptable salts, include but are not limited to acid addition salts that have been formed with the free amino groups of a protein.

The terms “prevent,” “preventing,” and “prevention” refer to the prevention of the onset, recurrence, or spread of a disease or disorder or of one or more symptoms thereof. The terms also encompass the inhibition or reduction of a symptom of the particular disease. Subjects with familial history of a disease in particular are candidates for preventive regimes in certain embodiments. In addition, subjects who have a history of recurring symptoms are also potential candidates for prevention. In this regard, the term “prevention” may be interchangeably used with the term “prophylactic treatment.”

A “prophylactically effective amount” of a compound is an amount sufficient to prevent a disease or disorder or prevent its recurrence. The term “prophylactically effective amount” can encompass an amount that improves overall prophylaxis or enhances the prophylactic efficacy of another prophylactic agent.

The term “subject” is defined herein to include animals such as mammals, including, but not limited to, primates (e.g., humans), cows, sheep, goats, horses, dogs, cats, rabbits, rats, mice and the like. In specific embodiments, the subject is a human.

As used herein, and unless otherwise specified, the phrase “therapeutically effective amount” a compound means an amount sufficient to provide a therapeutic benefit in the treatment or management of a disease or disorder, or to delay or minimize one or more symptoms associated with the disease or disorder. The phrase “therapeutically effective amount” can encompass an amount that improves overall therapy, reduces or avoids symptoms or causes of disease or disorder, or enhances the therapeutic efficacy of another therapeutic agent.

The terms “treat,” “treating,” and “treatment” refer to the eradication or amelioration of a disease or disorder, or of one or more symptoms associated with the disease or disorder. In certain embodiments, the terms refer to minimizing the spread or worsening of the disease or disorder by the administration of one or more prophylactic or therapeutic agents to a subject with such disease or disorder. In some embodiments the terms refer to the administration of a compound of formula I, with or without one or more additional active agent(s), after the onset of symptoms of a particular disease.

“Transdermal” relates to, being, or supplying a medication in a form for absorption through the skin into the bloodstream.

### Compounds

The compounds of the present invention are modified indole compounds that have the following common formula:

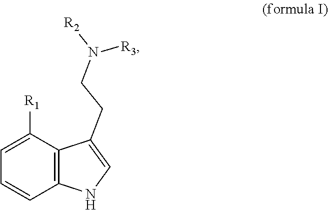

wherein
   R₁=COOH, CONH₂, CH₂OH, CH₂OCOCH₃, CH₂OCH₃, CH₂NH₂, CH₂CONH₂, CH₂NHSO₂CH₃, pyrazol, triazol, tetrazol, imidazol, or other nitrogen-containing, 5-membered heterocyclic moieties attached at either a carbon or nitrogen atom;
   R₂=H, CₙH₂ₙ₊₁, wherein n=1 to 12; and
   R₃=H, CₘH₂ₘ₊₁, wherein m=1 to 12.

In some embodiments, R₂ and R₃ are both H. In some embodiments, neither R₂ nor R₃ are H. In some embodiments, one of R₂ and R₃ is H and the other of R₂ and R₃ is not H.

In some embodiments, R₂ and R₃ are both CH₃. In some embodiments, neither R₂ nor R₃ are CH₃. In some embodiments, one of R₂ and R₃ is CH₃ and the other of R₂ and R₃ is not CH₃.

In some embodiments, R₂ and R₃ are both C₂H₅. In some embodiments, neither R₂ nor R₃ are C₂H₅. In some embodiments, one of R₂ and R₃ is C₂H₅ and the other of R₂ and R₃ is not C₂H₅.

In some embodiments, R₂ and R₃ are both C₃H₇. In some embodiments, neither R₂ nor R₃ are C₃H₇. In some embodiments, one of R₂ and R₃ is C₃H₇ and the other of R₂ and R₃ is not C₃H₇.

In some embodiments, R₂ and R₃ are both C₄H₉. In some embodiments, neither R₂ nor R₃ are C₄H₉. In some embodiments, one of R₂ and R₃ is C₄H₉ and the other of R₂ and R₃ is not C₄H₉.

In some embodiments, R₂ and R₃ are both C₅H₁₁. In some embodiments, neither R₂ nor R₃ are CH₁₁. In some embodiments, one of R₂ and R₃ is C₅H₁₁ and the other of R₂ and R₃ is not C₅H₁₁.

In some embodiments, R₂ and R₃ are both C₆H₁₃. In some embodiments, neither R₂ nor R₃ are C₆H₁₃. In some embodiments, one of R₂ and R₃ is C₆H₁₃ and the other of R₂ and R₃ is not C₆H₁₃.

In some embodiments, R₂ and R₃ are both C₇H₁₅. In some embodiments, neither R₂ nor R₃ are C₇H₁₅. In some embodiments, one of R₂ and R₃ is C₇H₁₅ and the other of R₂ and R₃ is not C₇H₁₅.

In some embodiments, R₂ and R₃ are both C₈H₁₇. In some embodiments, neither R₂ nor R₃ are C₈H₁₇. In some embodiments, one of R₂ and R₃ is C₈H₁₇ and the other of R₂ and R₃ is not C₈H₁₇.

In some embodiments, R₂ and R₃ are both C₉H₁₉. In some embodiments, neither R₂ nor R₃ are CoH₁₉. In some embodiments, one of R₂ and R₃ is C₉H₁₉ and the other of R₂ and R₃ is not CoH₁₉.

In some embodiments, R₂ and R₃ are both C₁₀H₂₁. In some embodiments, neither R₂ nor R₃ are C₁₀H₂₁. In some embodiments, one of R₂ and R₃ is C₁₀H₂₁ and the other of R₂ and R₃ is not C₁₀H₂₁.

In some embodiments, R₂ and R₃ are both C₁₁H₂₃. In some embodiments, neither R₂ nor R₃ are C₁₁H₂₃. In some embodiments, one of R₂ and R₃ is C₁₁H₂₃ and the other of R₂ and R₃ is not C₁₁H₂₃.

In some embodiments, R₂ and R₃ are both C₁₂H₂₅. In some embodiments, neither R₂ nor R₃ are C₁₂H₂₅. In some embodiments, one of R₂ and R₃ is C₁₂H₂₅ and the other of R₂ and R₃ is not C₁₂H₂₅.

In some embodiments, R₁ is a pyrazol, triazol, tetrazol, or imidazole moiety.

In some embodiments, the compounds are selected from the group consisting of:
(3-(2-(dimethylamino)ethyl)-1H-indol-4-yl)methanol;
3-(2-(dimethylamino)ethyl)-1H-indole-4-carboxylic acid;
3-(2-(dimethylamino)ethyl)-1H-indole-4-carboxamide;
(3-(2-(dimethylamino)ethyl)-1H-indol-4-yl)methyl acetate;
2-(4-(methoxymethyl)-1H-indol-3-yl)-N,N-dimethylethan-1-amine;
2-(4-(1H-pyrazol-5-yl)-1H-indol-3-yl)-N,N-dimethylethan-1-amine;
2-(4-((1H-1,2,4-triazol-1-yl)methyl)-1H-indol-3-yl)-N,N-dimethylethan-1-amine;
2-(4-((1H-1,2,4-triazol-1-yl)methyl)-1H-indol-3-yl)-N,N-dimethylethan-1-amine;
2-(4-(aminomethyl)-1H-indol-3-yl)-N,N-dimethylethan-1-amine;
N-((3-(2-(dimethylamino)ethyl)-1H-indol-4-yl)methyl) acetamide;
2-(4-(1H-tetrazol-5-yl)-1H-indol-3-yl)-N,N-dimethylethan-1-amine; and
N-((3-(2-(dimethylamino)ethyl)-1H-indol-4-yl)methyl)methanesulfonamide.

Additional examples of compounds within formula I include

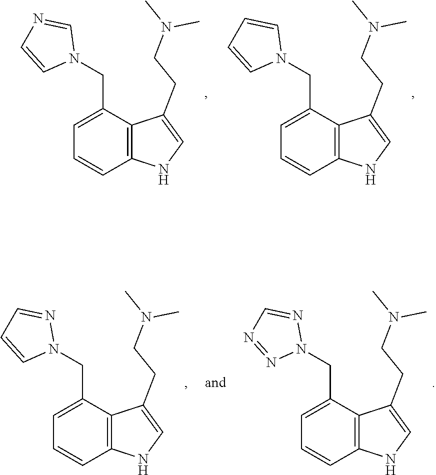

The compounds of the present invention may be in the form of Formula I or a salt thereof. In some embodiments, the salt is a pharmaceutically acceptable salt. In some embodiments, the salt is a tartrate salt, a maleate salt, a succinate salt, or a fumarate salt. In some embodiments, the salt is in a crystalline form.

### Formulations

The compounds of the present invention may be the active ingredient in, or one of a plurality of active ingredients in, formulations or medicaments that are designed to deliver these compounds. The formulations or medicaments may, for example, be applied transdermally and be in the form of sprayable liquids, gels, creams, lotions, ointments, transdermal patches and the like.

Alternatively they may be delivered orally. Oral formulations may be manufactured in the form of tablets, capsules, soft gels, strips, and oral patches.

Alternatively they may be delivered, intranasally. Intranasal administration may, for example, be in the form of a mist that is delivered to the nasal cavity.

In some embodiments, a compound of formula I is co-administered with one or more therapeutic agents. Co-administration may be simultaneously or sequentially. The co-administered agent may be a monoamine oxidase inhibitor (“MAOI”). MAOIs include, but are not limited to, harmala alkaloids, harmine, harmane, harmaline, hydrazine, iproniazid, isocarboxazid, nialamide, phenelzine, hydracarbazine, tranylcypromine, bifemelane, moclobemide, pirlindole, toloxatone, rasagiline, selegiline, safinamide, and other reversible inhibitors of monoamine oxidase A (RIMAs).

In another embodiment, a compound from another class of neurologically active agents is co-administered with a compound of formula I, thereby providing for a synergistic therapeutic effect. Other neurologically active agents include, but are not limited to, those compounds that fall into the following classes: antipsychotics, antidepressants, anxiolytics, stimulants, reuptake inhibitors (SSRI or SSNRI), cognitive-enhancing agents, tricyclic antidepressants, mood stabilizers, NMDA antagonists and 5-HT antagonists.

In some embodiments, a compound of formula I is co-administered with one or more therapeutic agents to reduce substance abuse. For the treatment of opioid addiction, other co-administered compounds can include, but are not limited to: methadone, buprenorphine, naloxone, naltrexone, and the like. For the treatment of alcoholism, other co-administered compounds can include, but are not limited to: ethyl alcohol, disulfiram, naltrexone, acamprosate, benzodiazepines, and the like. For the treatment of nicotine addiction, other co-administered compounds can include, but are not limited to: low dose nicotine, Bupropion, Varenicline and the like.

The formulations of the present invention may also comprise one or more excipients. Thus, in some embodiments, the present invention is directed to a composition or medicant that comprises a compound of formula I and one or more excipients. These excipients, may for example, act as one or more of diluents, binders, disintegrants, lubricants, coatings or films; and coloring agents. The formulations that contain these excipients may be designed for immediate release, controlled release or extended release.

### Uses

The present invention also the provides the compounds of formula I for use in the treatment, management, or prevention of a neurological, mood, or abuse disorder or disease. The disorder may, for example, be depression, central nervous system inflammation, addiction, headache, or dementia, or disorders of cognition and memory.

These products may be given prophylactically or therapeutically to subjects in need there of and be given in prophylactically effective amounts or therapeutically effective amounts. Methods for administering these compounds include, but are not limited to: orally, intranasally, through a transdermal route, intravenously, or through any other route that a person of ordinary skill in the art would deem effective for delivery of a DMT salt to subject.

The compounds of the present invention may, for example, be prepared from indole itself or related indole derived compounds such as those identified in examples 1 to 12 of this disclosure. Some of these methods begin with known indole derived compounds, while others begin with novel compounds disclosed herein.

### Examples

Computational binding scores were assessed for eleven compounds within formula I for the following relevant receptors: 6WGT (5-HT_{2A}), 5TVN (5-HT_{2B}), 4IAR (5-HT_{2C}), 5HK2 (σ-1), 4IAR (5-HT_{1B}), and 6HIN (5-HT₃). The results shown in Tables IA and IB predict effective binding for each compound.

TABLE IA

| | | | | 5TVN | 4IAR |
|---|---|---|---|---|---|
| | | | 6WGT (5- | (5- | (5- |
| Ex. No. | Structure | Name | HT_{2A}) | HT_{2B}) | HT_{2C}) |
| 1 | 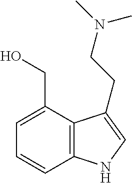 [SMILES: CN(C)CCc1c[nH]c2cccc(CO)c12] | (3-(2- (dimethylamino)ethyl)- 1H-indol-4-yl)methanol | −6.39 | −7.44 | −9.14 |
| 2 | 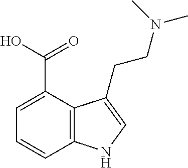 [SMILES: CN(C)CCc1c[nH]c2cccc(C(=O)O)c12] | 3-(2- (dimethylamino)ethyl)- 1H-indole-4-carboxylic acid | −10.39 | −6.95 | −9.88 |
| 3 | 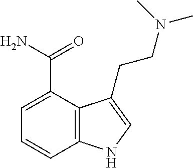 [SMILES: CN(C)CCc1c[nH]c2cccc(C(N)=O)c12] | 3-(2- (dimethylamino)ethyl)- 1H-indole-4-carboxamide | −7.57 | −6.41 | −9.76 |
| 4 | 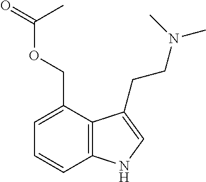 [SMILES: CC(=O)OCc1cccc2[nH]cc(CCN(C)C)c12] | (3-(2- (dimethylamino)ethyl)- 1H-indol-4-yl)methyl acetate | −9.97 | −6.74 | −8.63 |
| 5 | 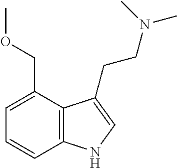 [SMILES: COCc1cccc2[nH]cc(CCN(C)C)c12] | 2-(4-(methoxymethyl)-1H- indol-3-yl)-N,N- dimethylethan-1-amine | −9.52 | −6.64 | −7.01 |
| 6 | 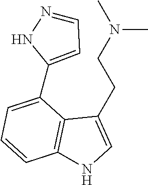 [SMILES: CN(C)CCc1c[nH]c2cccc(-c3ccn[nH]3)c12] | 2-(4-(1H-pyrazol-5-yl)- 1H-indol-3-y1)-N,N- dimethylethan-1-amine | −10.65 | −7.71 | −10.33 |
| 7 | 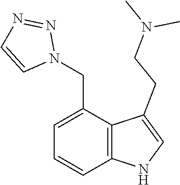 [SMILES: CN(C)CCc1c[nH]c2cccc(Cn3ccnn3)c12] | 2-(4-((1H-1,2,4-triazol-1- yl)methyl)-1H-indol-3-yl)- N,N-dimethylethan-1- amine | −8.89 | −7.05 | −6.83 |
| 8 | 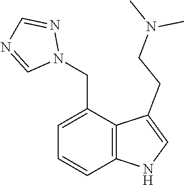 [SMILES: CN(C)CCc1c[nH]c2cccc(Cn3cncn3)c12] | 2-(4-((1H-1,2,4-triazol-1- yl)methyl)-1H-indol-3-yl)- N,N-dimethylethan-1- amine | −11.32 | −7.29 | −9.2 |
| 9 | 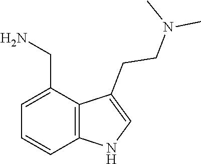 [SMILES: CN(C)CCc1c[nH]c2cccc(CN)c12] | 2-(4-(aminomethyl)-1H- indol-3-yl)-N,N- dimethylethan-1-amine | >−6.39 | −7.31 | −8.96 |
| 10 | 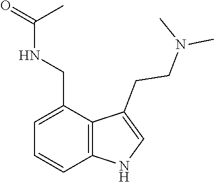 [SMILES: CC(=O)NCc1cccc2[nH]cc(CCN(C)C)c12] | N-((3-(2- (dimethylamino)ethyl)- 1H-indol-4- yl)methyl)acetamide | −9.46 | −6.72 | −9.1 |
| 11 | 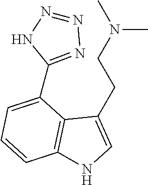 [SMILES: CN(C)CCc1c[nH]c2cccc(-c3nnn[nH]3)c12] | 2-(4-(1H-tetrazol-5-yl)- 1H-indol-3-y1)-N,N- dimethylethan-1-amine | −10.31 | −7.3 | −10.4 |

TABLE 1B

| | | | | | 6HIN |
|---|---|---|---|---|---|
| | | | 5HK2 | 4IAR | (5- |
| Ex. No. | Structure | Name | (σ-1) | (5-HT_{1B}) | HT₃) |
| 1 | 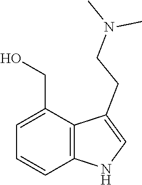 [SMILES: CN(C)CCc1c[nH]c2cccc(CO)c12] | (3-(2- (dimethylamino)ethyl)- 1H-indol-4-yl)methanol | −8.12 | −7.36 | −13.21 |
| 2 | 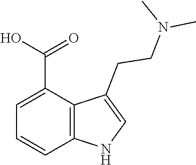 [SMILES: CN(C)CCc1c[nH]c2cccc(C(=O)O)c12] | 3-(2- (dimethylamino)ethyl)- 1H-indole-4-carboxylic acid | −8.49 | −8.32 | −10.68 |
| 3 | 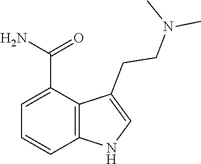 [SMILES: CN(C)CCc1c[nH]c2cccc(C(N)=O)c12] | 3-(2- (dimethylamino)ethyl)- 1H-indole-4-carboxamide | −10.96 | −7.59 | −9.52 |
| 4 | 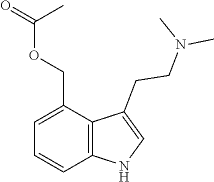 [SMILES: CC(=O)OCc1cccc2[nH]cc(CCN(C)C)c12] | (3-(2- (dimethylamino)ethyl)- 1H-indol-4-yl)methyl acetate | −9.05 | >−5.76 | −12.1 |
| 5 | 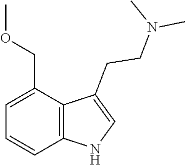 [SMILES: COCc1cccc2[nH]cc(CCN(C)C)c12] | 2-(4-(methoxymethyl)-1H- indol-3-yl)-N,N- dimethylethan-1-amine | −11.56 | −7.46 | −9.75 |
| 6 | 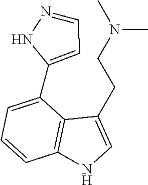 [SMILES: CN(C)CCc1c[nH]c2cccc(-c3ccn[nH]3)c12] | 2-(4-(1H-pyrazol-5-yl)- 1H-indol-3-yl)-N,N- dimethylethan-1-amine | −9.41 | −6.58 | −4.55 |
| 7 | 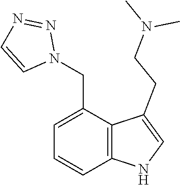 [SMILES: CN(C)CCc1c[nH]c2cccc(Cn3ccnn3)c12] | 2-(4-((1H-1,2,4-triazol-1- yl)methyl)-1H-indol-3-yl)- N,N-dimethylethan-1- amine | −10.13 | −6.23 | −13.14 |
| 8 | 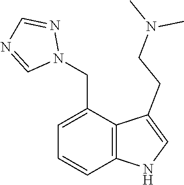 [SMILES: CN(C)CCc1c[nH]c2cccc(Cn3cncn3)c12] | 2-(4-((1H-1,2,4-triazol-1- yl)methyl)-1H-indol-3-yl)- N,N-dimethylethan-1- amine | −9.19 | −6.53 | −3.17 |
| 9 | 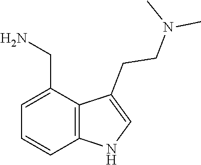 [SMILES: CN(C)CCc1c[nH]c2cccc(CN)c12] | 2-(4-(aminomethyl)-1H- indol-3-yl)-N,N- dimethylethan-1-amine | −8.98 | −8.73 | −9.48 |
| 10 | 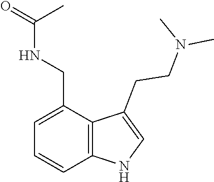 [SMILES: CC(=O)NCc1cccc2[nH]cc(CCN(C)C)c12] | N-((3-(2- (dimethylamino)ethyl)- 1H-indol-4- yl)methyl)acetamide | −8.82 | −7.51 | −11.72 |
| 11 | 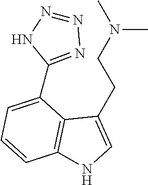 [SMILES: CN(C)CCc1c[nH]c2cccc(-c3nnn[nH]3)c12] | 2-(4-(1H-tetrazol-5-yl)- 1H-indol-3-yl)-N,N- dimethylethan-1-amine | −7.28 | −6.65 | −1.71 |

Below are eleven prophetic examples that describe how a person of ordinary skill in the art may synthesize the compounds identified in Table IA and Table IB, and a twelfth example that describes how one may synthesize N-((3-(2-(dimethylamino)ethyl)-1H-indol-4-yl)methyl)methanesulfonamide.

### Example 1: (3-(2-(dimethylamino)ethyl)-1H-indol-4-yl)methanol

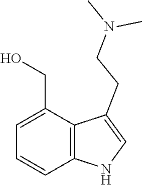 [SMILES: CN(C)CCc1c[nH]c2cccc(CO)c12]

Procedure: The benzyl alcohol will be prepared through the Speeter-Anthony Tryptamine Synthesis. Indole-4-carbaldehyde will be treated with oxalyl chloride followed by the appropriate amine to give the glyoxal amide which is then reduced to the tryptamine using lithium aluminum hydride.

### Example 2: 3-(2-(dimethylamino)ethyl)-1H-indole-4-carboxylic acid

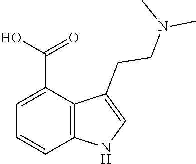 [SMILES: CN(C)CCc1c[nH]c2cccc(C(=O)O)c12]

Procedure: 3-(2-(dimethylamino)ethyl)-1H-indole-4-carboxylic acid will be made from indole-4-carboxaldehyde. The aldehyde will be acetal protected using dilute acid and ethylene glycol. The material will then be subjected to Speeter-Anthony Tryptamine Synthesis to afford the tertiary amine. The acetal protecting group will be removed under aqueous acidic conditions and then the aldehyde will be oxidized to the carboxylic acid under Pinnick Oxidation conditions using sodium chlorite, monosodium phosphate, and 2-methyl-2-butene.

### Example 3: 3-(2-(dimethylamino)ethyl)-1H-indole-4-carboxamide

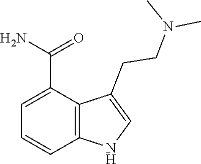 [SMILES: CN(C)CCc1c[nH]c2cccc(C(N)=O)c12]

Procedure: The amide will be synthesized by treating 3-(2-(dimethylamino)ethyl)-1H-indole-4-carboxylic acid with thionyl chloride to give the acid chloride. Bubbling ammonia through a solution of the acid chloride will provide the appropriate amide.

### Example 4: (3-(2-(dimethylamino)ethyl)-1H-indol-4-yl)methyl acetate

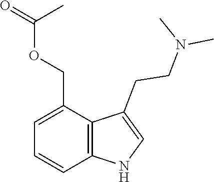 [SMILES: CC(=O)OCc1cccc2[nH]cc(CCN(C)C)c12]

Procedure: Acetic acid will be activated with 1-Ethyl-3-(3-dimethylaminopropyl) carbodiimide and catalytic dimethylaminopyridine in tetrahydrofuran. A solution of (3-(2-(dimethylamino)ethyl)-1H-indol-4-yl)methanol in tetrahydrofuran will be added dropwise to form the final product as a benzyl ester.

### Example 5:2-(4-(methoxymethyl)-1H-indol-3-yl)-N,N-dimethylethan-1-amine

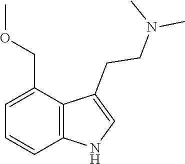 [SMILES: COCc1cccc2[nH]cc(CCN(C)C)c12]

Procedure: The indole nitrogen of Indole-4-carboxaldehyde will be protected with di-tert-butyl dicarbonate, triethylamine, and catalytic dimethylaminopyridine. The aldehyde at the 4-position will be reduced to the alcohol using sodium borohydride and then converted to the alkyl bromide using phosphorus tribromide. Sodium methoxide will be added to displace the bromide and make the methyl ether.

Finally, the benzyl ether will be treated with oxalyl chloride and then dimethylamine to make the glyoxal amide followed by a reduction with lithium aluminum hydride to give the final product.

### Example 6:2-(4-(1H-pyrazol-5-yl)-1H-indol-3-yl)-N,N-dimethylethan-1-amine

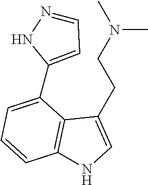 [SMILES: CN(C)CCc1c[nH]c2cccc(-c3ccn[nH]3)c12]

### Procedure: 2-(4-(1H-pyrazol-5-yl)-1H-indol-3-yl)-N,N-dimethylethan-1-amine

Will be prepared from indole-4-carboxaldehyde. The aldehyde will be converted to the ketone by addition of methyl magnesium bromide and then oxidation of the alcohol using Dess-Martin periodinane. The ketone is condensed with N,N-Dimethylformamide dimethyl acetal and then cyclized using hydrazine in glacial acetic acid.

### Example 7:2-(4-((1H-1,2,3-triazol-1-yl)methyl)-1H-indol-3-yl)-N,N-dimethylethan-1-amine

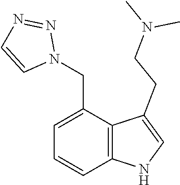 [SMILES: CN(C)CCc1c[nH]c2cccc(Cn3ccnn3)c12]

Procedure: The indole nitrogen of Indole-4-carboxaldehyde will be protected with di-tert-butyl dicarbonate, triethylamine, and catalytic dimethylaminopyridine. The aldehyde at the 4-position will be reduced to the alcohol using sodium borohydride and then converted to the alkyl bromide using phosphorus tribromide. 1,2,3-Triazole will be deprotonated using potassium carbonate and then added to a solution of the alkyl bromide to substitute the benzyl triazole. Finally, the benzyl triazole will be treated with oxalyl chloride and then dimethylamine to make the glyoxal amide followed by a reduction with lithium aluminum hydride to give the final product.

### Example 8:2-(4-((1H-1,2,4-triazol-1-yl)methyl)-1H-indol-3-yl)-N,N-dimethylethan-1-amine

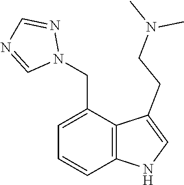 [SMILES: CN(C)CCc1c[nH]c2cccc(Cn3cncn3)c12]

Procedure: The indole nitrogen of Indole-4-carboxaldehyde will be protected with di-tert-butyl dicarbonate, triethylamine, and catalytic dimethylaminopyridine. The aldehyde at the 4-position will be reduced to the alcohol using sodium borohydride and then converted to the alkyl bromide using phosphorus tribromide. 1,2,4-Triazole will be deprotonated using potassium carbonate and then added to a solution of the alkyl bromide to substitute the benzyl triazole. Finally, the benzyl triazole will be treated with oxalyl chloride and then dimethylamine to make the glyoxal amide followed by a reduction with lithium aluminum hydride to give the final product.

### Example 9:2-(4-(aminomethyl)-1H-indol-3-yl)-N,N-dimethylethan-1-amine

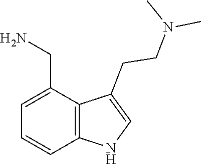 [SMILES: CN(C)CCc1c[nH]c2cccc(CN)c12]

Procedure: The benzyl amine will be prepared from indole-4-carbonitrile using the Speeter-Anthony Tryptamine Synthesis. Indole-4-carbonitrile will be treated with oxalyl chloride followed by dimethylamine to give the glyoxal amide which is then reduced to the tryptamine using lithium aluminum hydride.

### Example 10: N-((3-(2-(dimethylamino)ethyl)-1H-indol-4-yl)methyl)acetamide

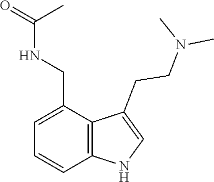 [SMILES: CC(=O)NCc1cccc2[nH]cc(CCN(C)C)c12]

Procedure: N-((3-(2-(dimethylamino)ethyl)-1H-indol-4-yl)methyl)acetamide will be prepared from 2-(4-(aminomethyl)-1H-indol-3-yl)-N,N-dimethylethan-1-amine. The benzyl amine will be treated with acetyl chloride (or an appropriate acid chloride) to afford the desired amide.

### Example 11:2-(4-(1H-tetrazol-5-yl)-1H-indol-3-yl)-N,N-dimethylethan-1-amine

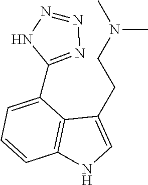 [SMILES: CN(C)CCc1c[nH]c2cccc(-c3nnn[nH]3)c12]

Procedure: The tetrazole will be prepared by treating indole-4-carbonitrile with sodium azide and ammonium chloride. The tryptamine sidechain will be installed using Speeter-Anthony tryptamine synthesis using oxalyl chloride, dimethylamine, and then reduction using lithium aluminum hydride to the tertiary amine.

### Example 12: N-((3-(2-(dimethylamino)ethyl)-1H-indol-4-yl)methyl)methanesulfonamide

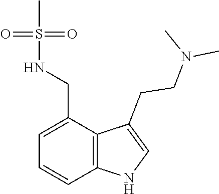 [SMILES: CN(C)CCc1c[nH]c2cccc(CNS(C)(=O)=O)c12]

Procedure: N-((3-(2-(dimethylamino)ethyl)-1H-indol-4-yl)methyl)methanesulfonamide will be prepared from 2-(4-(aminomethyl)-1H-indol-3-yl)-N,N-dimethylethan-1-amine. The benzyl amine will be treated with methane sulfonyl chloride (or any appropriate sulfonyl chloride) to afford the sulfonamide.

### Example 13:2-(4-(aminomethyl)-1H-indol-3-yl)-N,N-dimethylethan-1-amine

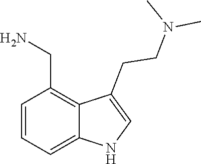 [SMILES: CN(C)CCc1c[nH]c2cccc(CN)c12]

Procedure: To a clean, dry reactor under an argon atmosphere was 2-(4-cyano-1H-indol-3-yl)-N,N-dimethyl-2-oxoacetamide (1.50 g, 6.22 mmol, 1 equiv) dissolved in 2-Me-THF (20.7 mL). The vessel was heated and a slurry was observed. To the slurry, LiAlH₄ (2.40 M in THF) (14.9 mL, 35.8 mmol, 5.75 equiv) was added dropwise. The thick mixture was heated and held overnight. The reaction was quenched with Glauber's Salt and filtered through celite. The pale yellow filtrate then concentrated under reduced pressure to give a yellow oil. The oil was dried under a blanket of nitrogen to afford the product as a pale yellow solid (1.30 g, 96%). 1H NMR (METHANOL-d₄, 600 MHz) δ 7.1-7.2 (m, 1H), 6.9-7.0 (m, 2H), 6.86 (d, 1H, J=7.3 Hz), 4.08 (s, 2H), 2.96 (t, 2H, J=1.0 Hz), 2.55 (t, 2H, J=1.0 Hz), 2.2-2.3 (m, 6H). ¹³C NMR (METHANOL-d₄, 151 MHz) δ 137.5, 133.9, 124.1, 122.6, 121.0, 117.8, 112.1, 110.2, 61.2, 44.1, 43.0, 24.8. HRMS calcd for C₁₃H₁₉N₃ ([M+H]⁺): 217.1579; found, 217.1577. 2-(4-(aminomethyl)-1H-indol-3-yl)-N,N-dimethylethan-1-amine was recovered.

### Example 14: N-((3-(2-(dimethylamino)ethyl)-1H-indol-4-yl)methyl)acetamide

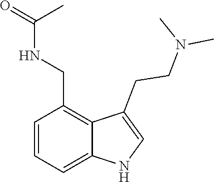 [SMILES: CC(=O)NCc1cccc2[nH]cc(CCN(C)C)c12]

Procedure: The amine (0.270 g, 1.23 mmol, 1 equiv) was dissolved in 2-Me-THF (3.19 mL) and cooled under argon. Acetyl chloride (0.107 mL, 1.37 mmol, 1.1 equiv) was added dropwise. The reaction was stirred overnight at room temperature. Ethyl acetate was added to the reaction. The organic phase was washed with water. The organic layer was dried over Na₂SO₄, filtered, and concentrated under reduced pressure to give a yellow oil. The oil was purified by silica-gel chromatography (100:0-90:10 DCM:MeOH/NH₃) to give a pale yellow solid (0.180, 56%). ¹H NMR (CHLOROFORM-d, 600 MHz) δ 8.61 (br s, 1H), 7.29 (d, 1H, J=8.0 Hz), 7.11 (t, 1H, J=7.6 Hz), 7.0-7.0 (m, 1H), 7.00 (d, 1H, J=7.3 Hz), 6.17 (br s, 1H), 4.78 (d, 2H, J=1.0 Hz), 3.02 (t, 2H, J=7.6 Hz), 2.63 (t, 2H, J=1.0 Hz), 2.30 (s, 6H), 1.98 (s, 3H). ¹³C NMR (CHLOROFORM-d, 151 MHz) δ 169.7, 137.1, 129.7, 125.0, 122.7, 121.9, 120.7, 114.3, 111.3, 61.2, 45.6, 42.5, 25.2, 23.2. HRMS calcd for C₁₅H₂₁N₃O ([M+H]⁺): 259.1685; found, 259.1684. N-((3-(2-(dimethylamino)ethyl)-1H-indol-4-yl)methyl) acetamide was recovered.

### Example 15: N-((3-(2-(dimethylamino)ethyl)-1H-indol-4-yl)methyl)methanesulfonamide

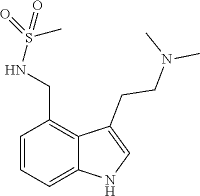 [SMILES: CN(C)CCc1c[nH]c2cccc(CNS(C)(=O)=O)c12]

Procedure: The amine (0.104 g, 0.479 mmol, 1 equiv) was dissolved in 2-Me-THF (1.23 and cooled under argon. Methanesulfonyl chloride (0.0822 mL, 0.718 mmol, 1.5 equiv) was added dropwise. The reaction was stirred overnight at room temperature. The precipitate was filtered off and washed with ether. The organic phase was washed with 2.00 M HCl, dried over Na₂SO₄, filtered, and concentrated under reduced pressure to give a yellow oil. The oil was purified by silica-gel chromatography (100:0-90:10 DCM:MeOH/NH₃) to give a pale tan solid (0.0609 g, 43%). ¹H NMR (CHLOROFORM-d, 600 MHz) δ 8.24 (br s, 1H), 7.33 (d, 1H, J=8.0 Hz), 7.13 (t, 1H, J=1.0 Hz), 7.08 (s, 1H), 7.07 (d, 1H, J=2.2 Hz), 4.69 (s, 2H), 3.10 (t, 2H, J=6.7 Hz), 2.71 (t, 2H, J=1.0 Hz), 2.68 (s, 3H), 2.27 (s, 6H). ¹³C NMR (CHLOROFORM-d, 151 MHz) δ 137.1, 129.6, 124.9, 123.0, 121.9, 121.9, 114.8, 111.7, 61.9, 45.7, 41.4, 25.6. HRMS calcd for C₁₄H₂₁N₃O₂S ([M+H]⁺): 295.1354; found, 295.1353. N-((3-(2-(dimethylamino)ethyl)-1H-indol-4-yl)methyl)methanesulfonamide was recovered.

### Example 16: (3-(2-(dimethylamino)ethyl)-1H-indol-4-yl)methyl acetate

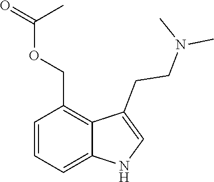 [SMILES: CC(=O)OCc1cccc2[nH]cc(CCN(C)C)c12]

Procedure: The amine (0.125 g, 0.575 mmol, 1 equiv) was dissolved in water (0.0104 mL) and acetic acid (0.0987 mL) and then cooled. NaNO₂ (0.0794 g, 1.15 mmol, 2 equiv) in a minimum amount of water was added dropwise to the reaction. The reaction was allowed to warm to room temperature and then neutralized with sat. NaHCO₃ and extracted with ether. The ether layer was dried over Na₂SO₄, filtered, and concentrated under reduced pressure. The oil was purified by silica-gel chromatography (100:0-90:10 DCM:MeOH/NH₃) to give a tan solid (0.0262 g, 21%). ¹H NMR (METHANOL-d₄, 600 MHz) δ 7.2-7.3 (m, 1H), 7.03 (s, 1H), 6.9-7.0 (m, 1H), 6.9-6.9 (m, 1H), 5.36 (s, 2H), 3.0-3.0 (m, 2H), 2.7-2.7 (m, 2H), 2.33 (s, 6H), 1.98 (s, 3H). HRMS calcd for C₁₅H₂₀N₂O₂ ([M+H]⁺): 260.1525; found, 260.1532. (3-(2-(dimethylamino)ethyl)-1H-indol-4-yl)methyl acetate was recovered.

### Example 17: (3-(2-(dimethylamino)ethyl)-1H-indol-4-yl)methanol

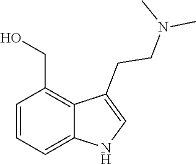 [SMILES: CN(C)CCc1c[nH]c2cccc(CO)c12]

Procedure: The amine (0.969 g, 4.46 mmol, 1 equiv) was dissolved in water (0.161 mL) and acetic acid (0.766 mL) and then cooled. NaNO₂ (0.615 g, 8.92 mmol, 1 equiv) in an excess amount of water was added dropwise to the reaction. The reaction was allowed to warm to room temperature and then neutralized with sat. NaHCO₃ and extracted with ether. The ether layer was dried over Na₂SO₄, filtered, and concentrated under reduced pressure to give a red oil. The oil was purified by silica-gel chromatography (100:0-90:10 DCM:MeOH/NH₃). (3-(2-(dimethylamino)ethyl)-1H-indol-4-yl)methanol was recovered.

1. A compound having the following formula: 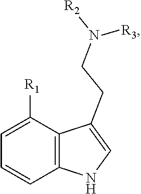
wherein
R₁=CONH₂, CH₂NH₂, CH₂CONH₂, CH₂NHSO₂CH₃, or a nitrogen-containing 5-membered heterocyclic moiety;
R₂=H or CH₂ₙ₊₁, wherein n=1 to 12; and
R₃=H or CₘH₂ₘ₊₁, wherein m=1 to 12.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. A method for treating, managing or preventing a neurological, mood, or abuse disorder or disease comprising administering a compound of claim 1 to a subject.

25. (canceled)

26. The compound of claim 1, wherein R₁=CH₂NHSO₂CH₃.

27. The compound of claim 26, wherein R₂=H; and R₃=H.

28. The compound of claim 26, wherein R₂=CH₂ₙ₊₁, wherein n=1; and R₃=CₘH₂ₘ₊₁, wherein m=1.

29. The compound of claim 26, wherein R₂=CₙH₂ₙ₊₁, wherein n=2; and R₃=CₘH₂ₘ₊₁, wherein m=2.

30. The compound of claim 26, wherein R₂=CₙH₂ₙ₊₁, wherein n=3; and R₃=CₘH₂ₘ₊₁, wherein m=3.

31. A method for treating, managing or preventing a neurological, mood, or abuse disorder or disease comprising administering a compound of claim 26 to a subject.

32. The compound of claim 1, wherein R₁=CH₂CONH₂.

33. The compound of claim 32, wherein R₂=H; and R₃=H.

34. The compound of claim 32, wherein R₂=CₙH₂ₙ₊₁, wherein n=1; and R₃=CₘH₂ₘ₊₁, wherein m=1.

35. The compound of claim 32, wherein R₂=CₙH₂ₙ₊₁, wherein n=2; and R₃=CₘH₂ₘ₊₁, wherein m=2.

36. The compound of claim 32, wherein R₂=CₙH₂ₙ₊₁, wherein n=3; and R₃=CₘH₂ₘ₊₁, wherein m=3.

37. A method for treating, managing or preventing a neurological, mood, or abuse disorder or disease comprising administering a compound of claim 32 to a subject.

38. The compound of claim 1, wherein R₁=CH₂NH₂.

39. The compound of claim 38, wherein R₂=H; and R₃=H.

40. The compound of claim 38, wherein R₂=CₙH₂ₙ₊₁, wherein n=1; and R₃=CₘH₂ₘ₊₁, wherein m=1.

41. The compound of claim 38, wherein R₂=CₙH₂ₙ₊₁, wherein n=2; and R₃=CₘH₂ₘ₊₁, wherein m=2.

42. The compound of claim 38, wherein R₂=CₙH₂ₙ₊₁, wherein n=3; and R₃=CₘH₂ₘ₊₁, wherein m=3.

43. A method for treating, managing or preventing a neurological, mood, or abuse disorder or disease comprising administering a compound of claim 38 to a subject.
